(19) United States
(12) Patent Application Publication
(10) Pub. No.: US 2018/0165620 A1
(43) Pub. Date: Jun. 14, 2018

(54) RECIPE AND MENU CREATION BASED UPON INGREDIENT INFORMATION

(71) Applicant: Sage Dining Services, Inc., Lutherville, MD (US)

(72) Inventors: Jason Ross, Towson, MD (US); Michelle Rodriguez Call, Salt Lake City, UT (US)

(21) Appl. No.: 15/841,772

(22) Filed: Dec. 14, 2017

Related U.S. Application Data
(60) Provisional application No. 62/434,104, filed on Dec. 14, 2016.

(51) Int. Cl. G06Q 10/06; G16H 20/60; G06F 17/30; G01G 19/414; G06F 3/0482

(52) U.S. Cl. CPC G06Q 10/06316; G16H 20/60; G06Q 50/12; G01G 19/4146; G06F 3/0482; G06F 17/3056

(57) ABSTRACT

A computer implemented method for recipe and menu creation based upon ingredient information includes receiving, from a menu author, one or more menu configuration parameters and rendering a multi-dimensional grid with cell dimensions corresponding to the one or more menu configuration parameters. Input is received from the menu author, for each cell to the multi-dimensional grid specifying one or more recipes. Each of the recipes comprises at least one ingredient. Each ingredient has an associated ingredient scratch factor corresponding to the ingredient's processing characteristics. For each of the recipes, a recipe scratch factor is calculated based on the associated ingredient scratch factor corresponding to each ingredient in the recipe. A menu scratch factor is calculated by summing the recipe scratch factors, and it is determined whether the menu meets dietary guidelines. If the menu meets the dietary guidelines, the menu is displayed to users.

### CROSS REFERENCE TO RELATED APPLICATIONS

This application claims priority to U.S. Provisional Patent Application Ser. No. 62/434,104, filed on Dec. 14, 2016, the entire contents of which are hereby incorporated by reference herein.

### TECHNOLOGY FIELD

The present disclosure generally relates to a system and method for the creation of recipes and menus using information about their constituent ingredients' information.

### BACKGROUND

Cooking from scratch is currently seen as desirable over serving prepared foods, i.e. those manufactured in food processing facilities. It is currently easy to claim that a menu item is “made from scratch,” but such claims are not quantifiable and generally do not apply to the totality of the menu. Even the ingredients used in “scratch cooking” can themselves be made off-premises in a food manufacturing facility. The consumer and the food service professional lack a metric by which to measure the degree to which a menu is cooked “from scratch.” Moreover, food service providers lack a simple to use tool by which recipes and menus can be created in consideration of to what degree a claimed “from scratch” ingredient really is from scratch.

### SUMMARY

Embodiments of the present invention address and overcome one or more of the above shortcomings and drawbacks, by providing methods, systems, and apparatuses related to recipe and menu creation based upon ingredient information.

According to some embodiments, a computer implemented method for recipe and menu creation based upon ingredient information includes receiving, from a menu author, one or more menu configuration parameters and rendering a multi-dimensional grid with cell dimensions corresponding to the one or more menu configuration parameters. Input is received from the menu author, for each cell to the multi-dimensional grid specifying one or more recipes. Each of the recipes comprises at least one ingredient. Each ingredient has an associated ingredient scratch factor corresponding to the ingredient's processing characteristics. For each of the recipes, a recipe scratch factor is calculated based on the associated ingredient scratch factor corresponding to each ingredient in the recipe. A menu scratch factor is calculated by summing the recipe scratch factors, and it is determined whether the menu meets dietary guidelines. If the menu meets the dietary guidelines, the menu is displayed to users.

According to other embodiments, a computer-implemented method for recipe and menu creation based upon ingredient information includes receiving input for each cell to a multi-dimensional grid specifying one or more recipes. Each of the one or more recipes comprises at least one ingredient, each ingredient having an associated ingredient scratch factor corresponding to the ingredient's processing characteristics. For each of the recipes, a recipe scratch factor is calculated based on the associated ingredient scratch factor corresponding to each ingredient in the recipe. A menu scratch factor is calculated by summing the recipe scratch factors and, if the menu meets the dietary guidelines, the menu is saved.

According to other embodiments, a computer-implemented method for recipe and menu creation based upon ingredient information includes creating a menu comprising a plurality of recipes, wherein each recipe comprises at least one ingredient. Then, for each recipe in the menu, an ingredient scratch factor is calculated for each ingredient in the recipe based on the ingredient's processing characteristics. Next, for each recipe in the menu, a recipe scratch factor is calculated based on the ingredient scratch factor corresponding to each ingredient in the recipe. A menu scratch factor is calculated by summing the recipe scratch factors and, if the menu meets the dietary guidelines, the menu is displayed to one or more users.

Additional features and advantages of this disclosure will be made apparent from the following detailed description of illustrative embodiments that proceeds with reference to the accompanying drawings.

### BRIEF DESCRIPTION OF THE DRAWINGS

The foregoing and other aspects of the present invention are best understood from the following detailed description when read in connection with the accompanying drawings. For the purpose of illustrating the inventions described herein, there is shown in the drawings embodiments that are presently preferred, it being understood, however, that the inventions are not limited to the specific instrumentalities disclosed. Included in the drawings are the following Figures:

FIG. 1 illustrates a block diagram depicting the components of a recipe and menu creation system, according to embodiments described herein;

FIG. 2 illustrates a block diagram depicting the menu authoring process, in accordance with embodiments described herein;

FIG. 3 illustrates a block diagram depicting the recipe authoring process, in accordance with embodiments described herein;

FIG. 4 illustrates a block diagram depicting the ingredient authoring process, in accordance with embodiments described herein;

FIG. 5 illustrates a block diagram depicting the process of updating a menu scratch factor after a new recipe has been added, in accordance with embodiments described herein;

FIG. 6 illustrates a block diagram depicting the process of calculating a recipe scratch factor, in accordance with embodiments described herein;

FIG. 7 illustrates a block diagram depicting the process of updating an ingredient scratch factor, in accordance with embodiments described herein; and

FIG. 8 illustrates an example of a computing environment within which embodiments of the invention may be implemented.

### DETAILED DESCRIPTION OF EXEMPLARY EMBODIMENTS

The following disclosure describes the present invention according to several embodiments directed at methods, systems, and apparatuses related to creating and/or rating an entire menu which can measure the degree to which it is cooked “from scratch.” In an embodiment, the system can begin by ascribing numerical values to individual ingredients and then compiling ratings for recipes made from those ingredients. The system can then compile a rating for the entire menu being served from the list of recipes for the items on the menu.

A menu can be a collection of recipes and each recipe can be itself a collection of ingredients. Some ingredients can be raw or unprocessed commodities. Other ingredients can be fully processed foods, and some can be semi-processed. A menu may incorporate dozens of individual items, where each of which can have its own recipe. The kitchen can draw these recipes from a database of thousands of distinct recipes. The recipes themselves can draw from tens of thousands of ingredients.

The system can assign a numerical rating, on a scale from zero to one, to all of the ingredients used by a kitchen based upon how “from scratch” the ingredient is, which can be called the scratch factor. Once all of the ingredients in a recipe are thus numerically coded according to their scratch factor, the recipe can receive a numerical rating that is the average of its ingredients' ratings. This unique number is the recipe scratch factor. Once all of the recipes for all of the items on a menu have been rated, then the entire menu can be rated for a menu scratch factor, which can be the numerical average of the scratch factor for each of its individual recipe items.

The system and method described herein can provide food service professionals a means by which to construct menus that maximize the number of from scratch offerings, an attribute highly prized by consumers. A chef can select menu items from a large list of recipes based upon their individual recipe scratch factor scores, and by doing so maximize the consumer appeal of his/her menu. Consumers can benefit from the ability to compare menus based on their scratch factor, allowing them to select between competing menus based upon an attribute they find important: scratch cooking.

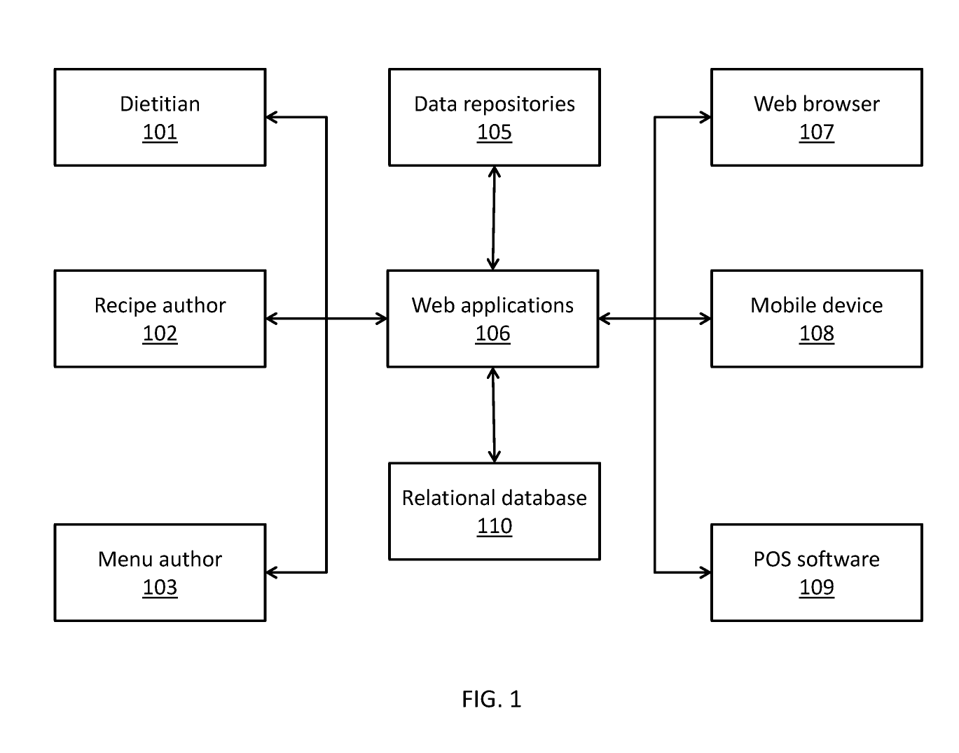

FIG. 1 illustrates a block diagram depicting the components of a recipe and menu creation system, according to embodiments described herein. A dietitian 101 can approve new ingredients and apply a series of guidelines to each new ingredient to determine the ingredient's scratch factor through the use of the system's web applications 106. Various methodologies may be used to determine each ingredient's scratch factor. For example, in one embodiment, raw or unprocessed commodities can be assigned the numerical value of one, which can be the highest level of scratch factor. Completely processed foods can be assigned the numerical value of zero. Semi-processed foods can be assigned a value in between one and zero depending on their level of processing. For example, eggs can have a value of one, bread can have a value of zero, and dried pasta, which can require cooking, can receive a value of one half.

In some embodiments, each ingredient's scratch factor is manually input by the dietitian 101. In other embodiments, the recipe and menu creation system may automatically determine the scratch factor, for example, based on a local or remote database of ingredient scratch factor data. For example, in some embodiments, the recipe and menu creation system may communicate with a cloud-based storage system (not shown in FIG. 1) to retrieve one or more files with the ingredient scratch factor data. In this way, ingredient scratch factor data can be centralized and accessible to multiple systems. Thus, updates can be made to the ingredient scratch factor data in a single location rather than requiring updates at each system. In one embodiment, updates to the ingredient scratch factor data are pushed out to each recipe and menu creation system as they are generated. In other embodiments, each recipe and menu creation system periodically retrieves the ingredient scratch factor data from the cloud-based system (e.g., upon startup or at a regular interval).

Returning to FIG. 1, recipe authors 102 can compose new recipes from approved ingredients. The ingredients used and the amount of each ingredient used can determine the recipe's scratch factor. In an embodiment, the recipe scratch factor can be determined by applying a weighted average of each ingredient's individual scratch factor. The weight applied to each ingredient may be based, for example, on the proportion of the ingredient in the recipe. For example, if 25% of a recipe is tomatoes, the weight associated with tomatoes may be set to 0.25 on a 1 point scale or 25 on a 100 point scale. Alternatively, the weight may be based on other factors such as the preferences of the dietitian 101 or popularity with customers. Additionally, in some embodiments, weighting values may incorporate a variety of factors (e.g., a combination of proportion of the ingredient in the recipe and preferences of the dietitian).

Continuing with reference to FIG. 1, menu authors 103 can use the system's web applications 106 to compose variable length menus from approved recipes contained within one or more recipe or menu repositories 105. In an embodiment, the average of all recipe scratch factor scores can become the menu's scratch factor. The relationships between the menus, recipes, and ingredients, when in use by the system during creation of a menu or recipe, can be stored in a relational database 110 that can be used to store connections between one or more of the items.

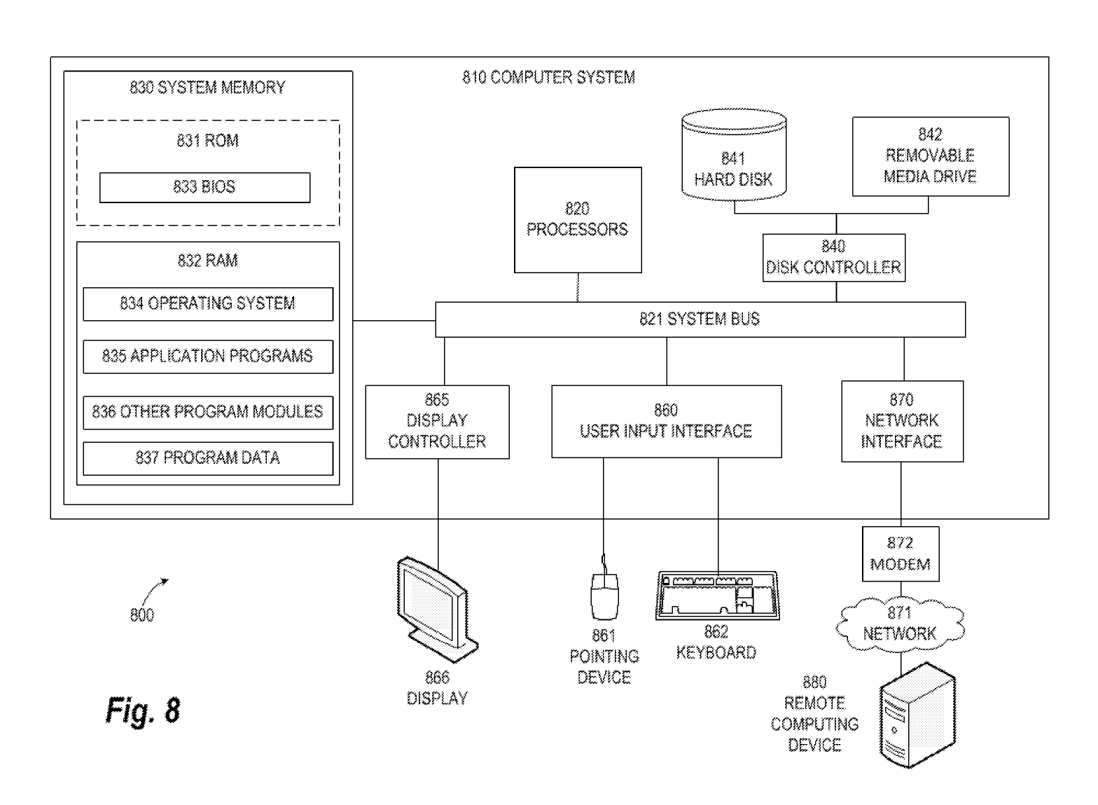

Ingredient, recipe, and menu information can be mediated and transmitted through users and inputters of the system through the system's web applications 106, which are described further in FIG. 8. Ingredient, recipe, and menu information can be stored in one or more data repositories 105. Information can be collected and displayed by the system through a web browser 107, mobile device 108, or point-of-service (POS) software 109, which can run on a tablet or station computer. The recipe and menu information can be used by customers and food service professionals to make choices about what to eat or serve and how to understand how much of the menu is objectively made from scratch.

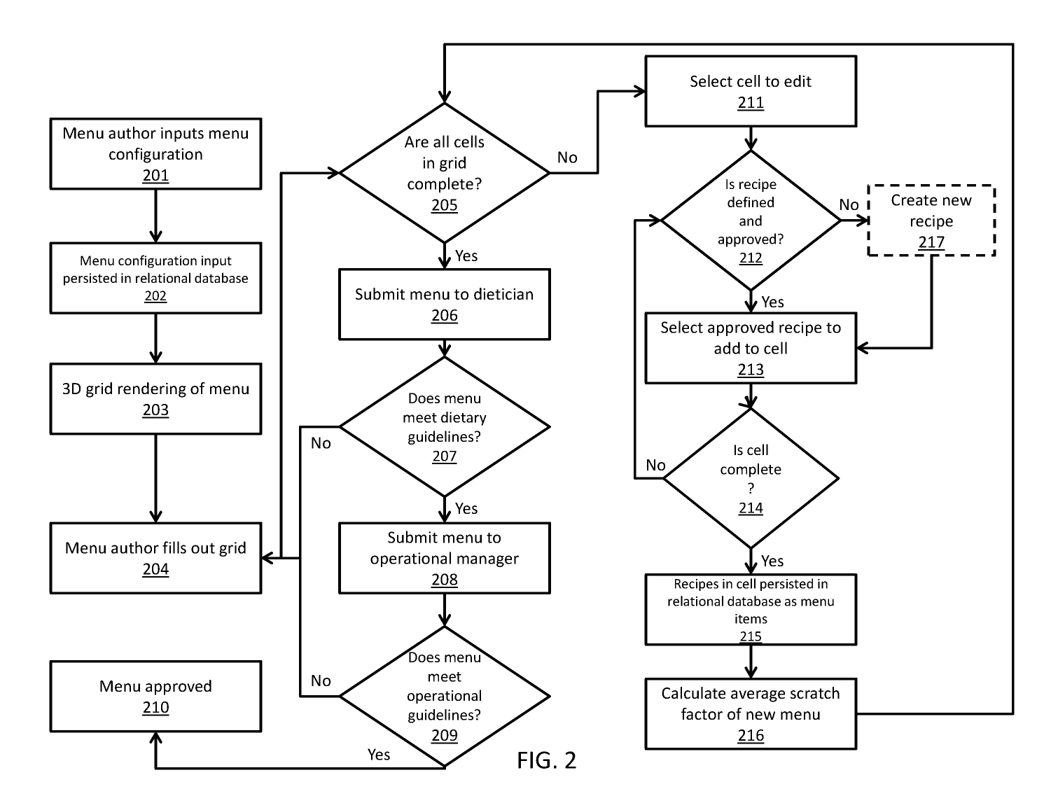

FIG. 2 illustrates a block diagram depicting the menu authoring process, in accordance with embodiments described herein. To begin the menu authoring process using the menu and recipe creation system, a menu author can author a new menu. The menu author can begin by inputting basic configuration parameters for the menu 201. These menu configuration parameters can include, for example, the number of meals in a day, number of serving days, and number of weeks for which meals are to be served, and whether different meals are to be served at different serving stations.

Once the menu configuration is created, it may be persisted in a database 202. In some embodiments, this database is a relational database such as MySQL. In other embodiments, menu configuration data can be stored in a No-SQL database which has a table equivalent structure. As is understood in the art, the term “No-SQL” is used to define a class of data stores that are non-relational in their design. There are various types of No-SQL databases which may be generally grouped according to their underlying data model. These groupings may include databases that use column-based data models (e.g., Cassandra), document-based data models (e.g., MongoDB), key-value based data models (e.g., Redis), and/or graph-based data models (e.g., Allego). Any type of No-SQL database may be used to implement the various embodiments described herein. For example, in one embodiment, MongoDB software is used to provide the underlying functionality of the database used by the systems described herein.

Using the menu configuration information, the system can then create a three dimensional rendering of a blank menu document 203. In an embodiment, the rendering can contain one or more cells corresponding to each serving station for every meal, day and week, according to the menu configuration parameters input by the menu author. Each cell of the three dimensional grid can correspond to a particular serving station or location, and can contain one or more recipes. In an embodiment, the three dimensional grid can be rendered in an HTML user interface, with the grid dimensions representing meal and station by day of the week by the week itself, which can represent a variable length menu schedule for the number of weeks defined in the configuration. The cells of the grid can hold menu items that can be selected by the menu author, and can take the form of drop downs or radio buttons. The menu author can then begin adding approved recipes to each cell and filling out the grid 204.

The system then determines if all cells in the grid are filled to completion 205. If the system determines that the cells in the grid are complete, the system submits the menu to a dietician for approval 206. In some embodiments, the contact information for the dietician can be associated with the menu as a system setting. In other embodiments, the system can select the dietician from a listing of dieticians based on factors such as the ingredients used on the menu. For example, for a vegetarian menu, a dietician that may be selected that specializes in vegetarian meals. The dietician can be notified of the menu, for example, via e-mail or a notification on his or her mobile device. The system can then solicit feedback from the dietician as to whether or not the menu meets all dietary guidelines, which can include ingredient, recipe, or menu scratch factor minimums 207. In some embodiments, the system may also facilitate a payment to the dietician in exchange for providing feedback to the menu. For example, the menu author may be charged a fee for using the system and a portion of this fee may be transferred to the dietician after receiving the feedback. Techniques for exchanging money between computing systems and banking accounts are generally known to one skilled in the art and, thus, these techniques are not described in detail herein.

As an alternative to using a dietician, in some embodiments, the system determines if the menu meets all dietary guidelines by automatically comparing the cell items to preset thresholds for one or more nutritional metrics, which can include ingredient, recipe, or menu scratch factor minimums. These thresholds may be set, for example, by the dietician or a remote entity. In some embodiments, a dietician may specify that the menu meets the dietary guidelines if the recipes in the menu collectively meet or exceed a specific menu scratch factor minimum value. For example, a dietician may set a requirement that the menu scratch factor must be at least 0.75 on 1 point scale. Thus, by comparing the menu scratch factor menu scratch factor minimum, the system can automatically determine whether the menu meets dietary guidelines.

As an alternative or supplement to focusing on the menu scratch factor menu, in some embodiments, the nutritional metric thresholds are set based on Dietary Reference Intakes (DRIs) such as Recommended Dietary Allowance (RDA), Adequate Intake (AI), and Tolerable Upper Intake Level (UL). As is generally understood in the art, entities such as the Institute of Medicine (TOM) of the National Academies of Sciences, Engineering, and Medicine provide standard nutrition recommendations. By using standard tables provided by such entity, the system may set the nutritional metric thresholds described herein. For example, if the RDA for folate is determined to be 400 micrograms per day, a nutritional metric threshold may be set that the overall menu must include at least this level of folate. The nutritional metric threshold may also be set on a per-meal basis. For example, by assuming that an individual consumes 3 meals per day, the nutritional metric threshold for the meal may be set by dividing the daily nutritional metric threshold by 3. To continue with the folate example, the nutritional metric threshold for each meal may be set as 400/3 micrograms per meal.

In some embodiments, the nutritional metric thresholds are customized for a particular individual. For example, in one embodiment, an individual's electronic medical record (EMR) is parsed to determine dietary restrictions specified by a physician. Based on these restrictions, the thresholds may be set accordingly. This process can also be repeated for a population of individuals (e.g., a school or nursing home) to consider whether the menu meets the dietary restrictions for each member of the population. Additionally, the thresholds may be used to ensure that known allergens are not used in the menu. For example, if an individual is known to have a nut allergy, the threshold for nut-based ingredients may be set 0.

If the system determines that the menu does not meet the desired dietary guidelines, the system can provide an error message to the menu author, and require the menu author to alter the contents of one or more cells in the grid with alternate recipes having different scratch factor values. If the menu does meet the dietary guidelines, the system can submit the menu to an operational manager for approval 208. An operational manager can be a restaurant executive chef, food court manager, or other administrative person with operational control over menus. The system, using input from the operational manager, can determine if the menu meets operational guidelines, which can also include scratch factor minimums 209. If the menu does not meet operational guidelines, the system can provide an error message to the menu author, and require the menu author to alter the contents of one or more cells in the grid with alternate recipes. If the menu meets operational guidelines, the menu can be approved for implementation 210. In an embodiment, the system can omit the steps of submitting the menu to an operational manager 208 and determining if operational guidelines are met 209, and can instead approve the menu based solely on whether the menu meets dietary guidelines 207.

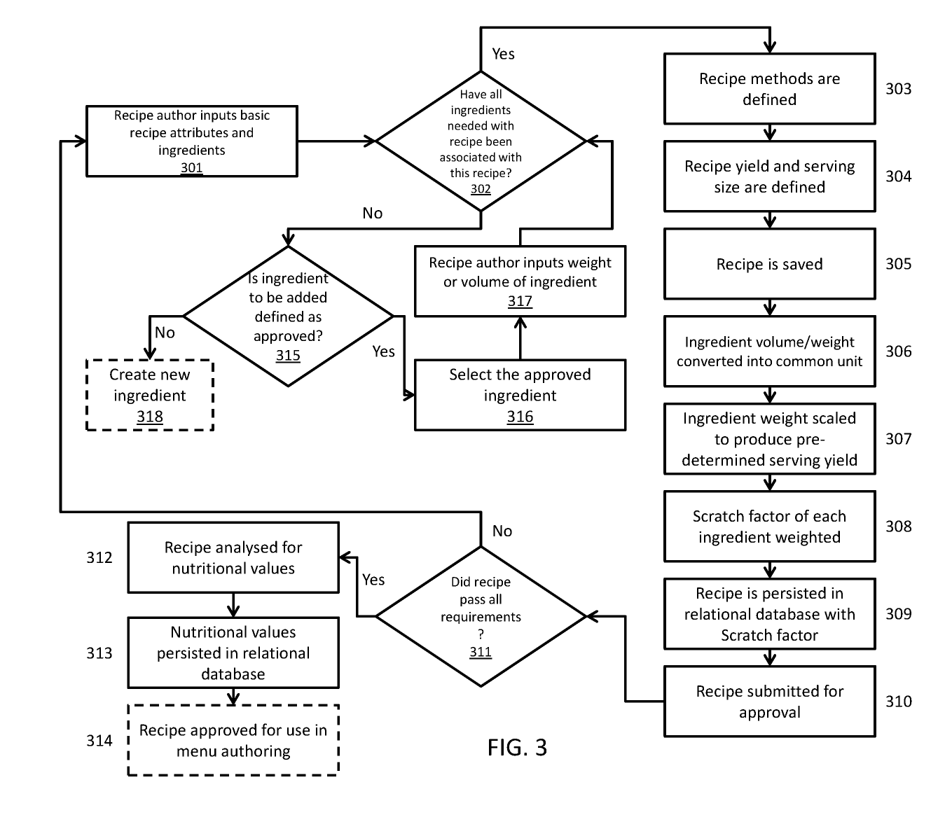

If the system determines that not all cells in the grid are complete (i.e., are missing one or more recipes), the system can highlight the incomplete cells. The menu author can first select a cell to edit 211. The system can then determine if the recipe the menu author desires to enter into the cell is defined and approved 212. If at any point the menu author desires to serve a recipe that is not approved, they may create and submit a new recipe for approval 217. The recipe creation process is further discussed in FIG. 3. Once the newly created recipe is approved, it may be added to the menu. If the desired recipe is defined and complete, the recipe can be added to the cell 213. The system can then do a second check to determine if the cell is complete 214. If the cell is not complete, the system can prompt the menu author to input additional information, and can check if the new recipe is defined and approved 212.

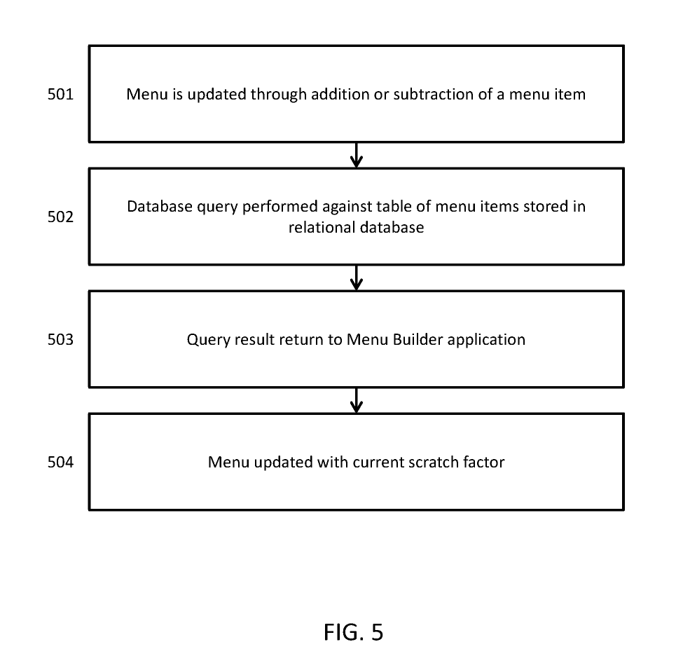

If the cell is determined to be complete 214, the recipes contained in the cell can be persisted in the relational database as menu items 215. Incorporating the new menu items, the system can calculate an updated scratch factor of the new menu 216. In an embodiment, the menu scratch factor can be calculated by averaging the scratch factor of each recipe in the menu. Updating the menu scratch factor is further described in FIG. 5. Once the new menu scratch factor is calculated, the system can repeat the step wherein the system determines if all cells in the grid are complete 205.

FIG. 3 illustrates a block diagram depicting the recipe authoring process, in accordance with embodiments described herein. The recipe authoring process can be initiated during the menu authoring process (as described in FIG. 2), or can be independently run by a recipe author at an alternate time. To begin, the recipe author can input basic recipe attributes, such as title, description, whether any special equipment is needed, and approved ingredients to the recipe 301. Additionally, as the recipe author selects each ingredient, they can input the amount of the ingredient needed for the recipe 317. In an embodiment, the recipe author can input the weight or volume of a particular ingredient in a unit of measure that is applicable to that particular ingredient. The system can then determine if all the ingredients needed with the recipe are actually associated with the recipe 302. In an embodiment, the system can use an external resource, such as a third-party database of recipes, to aid the determination of whether a recipe with a particular title is missing an ingredient or appears to have an incorrect amount of ingredient added to the recipe.

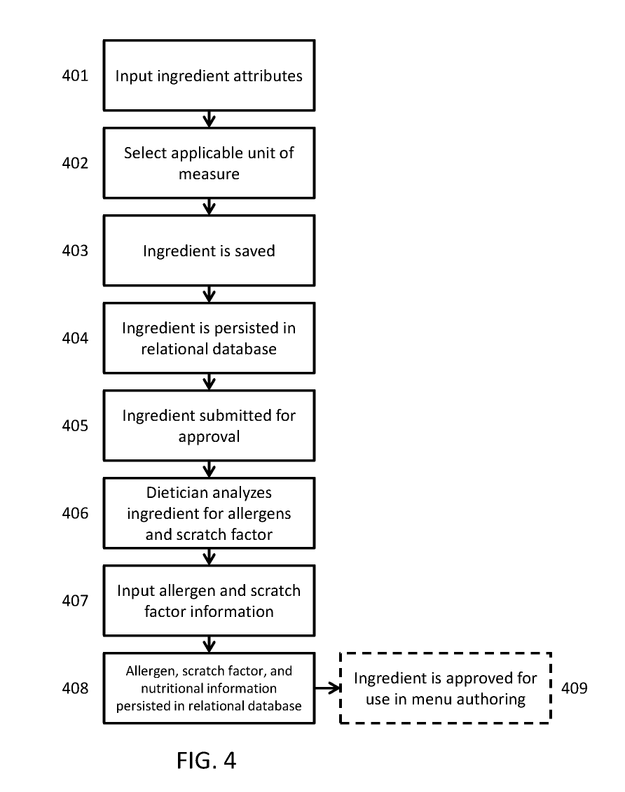

If the system determines that not all ingredients associated with the recipe are actually associated with the recipe, the system will prompt the recipe author to add an ingredient. Upon introduction of a new ingredient, the system will determine if the ingredient to be added is already defined as an approved ingredient 315. If not, the recipe author will be prompted to create a new ingredient 318. The ingredient creation process is further described in FIG. 4. If the ingredient is already approved, the system can select the approved ingredient 316, and prompt the user to input the weight or volume of the ingredient needed for the recipe 317.

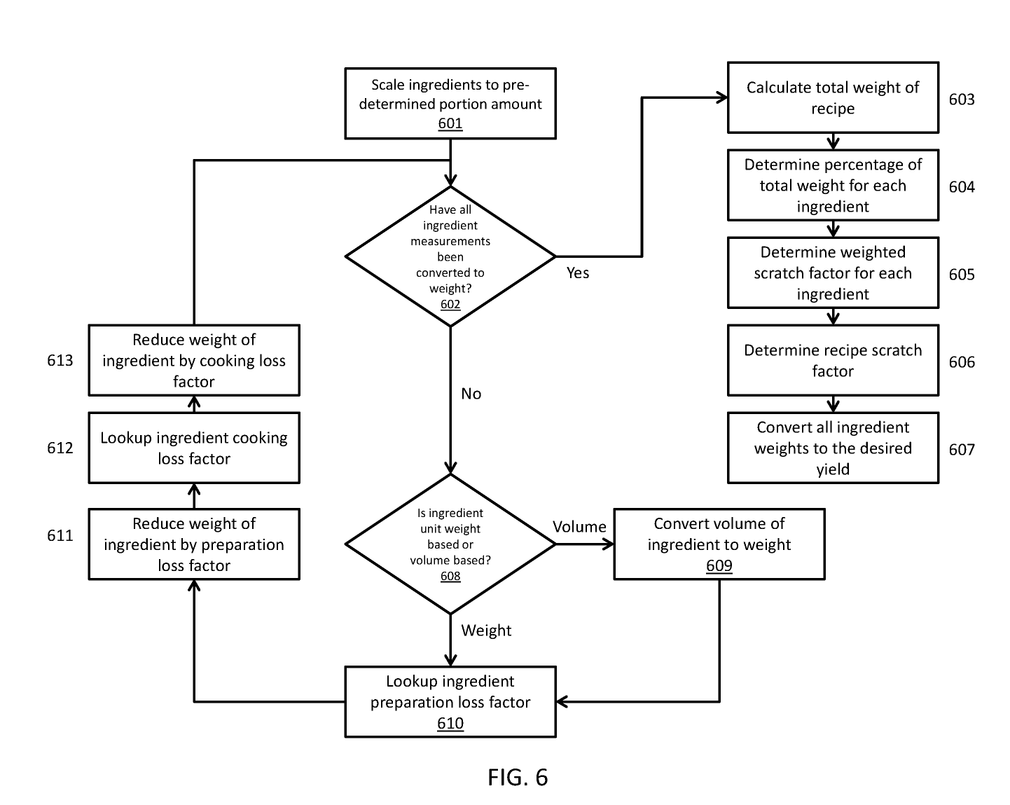

If the system determines that all the ingredients needed with the recipe are actually associated with the recipe, the system can allow the recipe author to define the recipe preparation and cooking methods 303. The recipe author can then define the yield and serving size that the recipe makes 304. The system can then save the completed recipe into the data repository 305. The system can then convert each recipe ingredient's weight or volume into a common unit of measure for weight 306. In an embodiment, the conversion can occur through the use of one or more lookup tables, and is described further in FIG. 6. The weight of each ingredient can then be scaled up or down to produce a pre-determined serving yield 307. In an embodiment, the serving yield can be fifty servings, and can factor loss of weight due to handling and cooking of the ingredient. The scratch factor of each ingredient can then be weighted 308. In an embodiment, the scratch factor can be weighted according to the percentage of weight the ingredient occupies in the finished recipe.

After the weighted ingredient scratch factor is calculated 308, the system can persist the recipe within the relational database, and can include the recipe's calculated scratch factor 309. In an embodiment, the recipe scratch factor can be calculated by summing the weighted ingredient scratch factors. After persistence, the recipe can be submitted to the system or the dietician for approval 310. The system can then determine if the recipe passes all dietary requirements 311. If the recipe does not pass all dietary requirements, the recipe author will be required to alter one or more of the basic recipe attributes 301 and repeat the recipe creation process.

If the recipe passes all requirements, the recipe can then be analyzed by the system for its nutritional values 312. The nutritional values can also be persisted in the relational database 313. After the values are persisted, the recipe can be approved for use in menu authoring 314. Approval can mean the recipe is introduced straight into the menu authoring process, if the recipe authoring process was initiated as part of the menu authoring process, or the recipe can be stored in the data repository for later use by a menu authoring process occurring at a subsequent time.

FIG. 4 illustrates a block diagram depicting the ingredient authoring process, in accordance with embodiments described herein. If at any time during the ingredient selection process used during the recipe authoring process, a recipe author finds the need to add an ingredient to the list of approved ingredients, they may create a new ingredient. To begin the ingredient authoring process, a recipe author can input the ingredient's attributes 401. Ingredient attributes can include the ingredient's name and its form (i.e., whole, sliced, chopped, ground, mashed, fresh, dried, cooked, etc.). The recipe author can then select the applicable units of measurement for that ingredient 402. The ingredient can then be saved 403 and persisted in the relational database 404. After persistence, the recipe author can then submit the ingredient for approval 405. During the approval process, a dietitian can then analyze the ingredient for allergens, and can determine an ingredient scratch factor score for the ingredient 406. The dietician can then input the allergen and scratch factor information regarding the ingredient into the system 407. The allergen, scratch factor, and nutritional information can then be persisted in the relational database 408. After persistence, the ingredient can be added to the list of approved ingredients for use in a recipe 409.

FIG. 5 illustrates a block diagram depicting the process of updating a menu scratch factor after a new recipe has been added, in accordance with embodiments described herein. To initiate the menu update process, the menu can be updated, either through addition or subtraction of a menu item (a recipe) 501. The system can then perform a database query against a table of menu items stored in the relational database 502. In an embodiment, the database query can select the average of the menu item column designated as the scratch factor column for all menu item rows associated with the active menu being updated. The result of the query can then be returned to the menu builder application 503, and can be displayed on the user interface as the updated aggregate menu scratch factor 504.

FIG. 6 illustrates a block diagram depicting the process of calculating a recipe scratch factor, in accordance with embodiments described herein. The process can begin by scaling the ingredients in a recipe to a pre-determined portion amount 601. In an embodiment, the portion amount can be 50 portions. The system can then determine if all ingredient measurements have been converted to weight 602. If the ingredient measurements have not been converted to weight, the system can determine if the ingredient units are weight based or volume based 608. If the ingredient units are volume based, the system can convert the volume unit of the ingredient to a weight unit 609. In an embodiment, the system can convert volume to weight using one or more lookup tables stored in the relational database.

If the ingredient units are weight based, or if the system has converted the volume units into weight units, the system can look up the ingredient's preparation loss factor 610, which can be stored in a table in the relational database. The preparation loss factor can represent the amount of weight loss that can occur when the ingredient is prepared for cooking (for instance, for an ingredient such as carrots that may be bought with stems, the preparation factor can consider the weight reduction of the ingredient if the stems are removed). The system can then reduce the weight of the ingredient by the preparation loss factor 611. The system can then look up the ingredient cooking loss factor 612, which can represent the weight loss to the ingredient caused by a cooking process. The system can then further reduce the weight of the ingredient by the cooking loss factor 613, and re-determine if all ingredient measurements have been converted to weight 602.

If all ingredient units of measure have been converted to weight equivalents, the system can then add all of the ingredients' weights to determine the total weight of the finished recipe 603. The system can then divide each ingredient's weight by the total recipe weight to determine the percentage of total weight for each ingredient 604. The system can then then multiply each ingredient's scratch factor by its percentage of total weight to determine the weighted ingredient scratch factor 605. The system can then add all of the weighted ingredient scratch factors to determine the overall recipe scratch factor 606. The system can then finish the process by converting all ingredient weights to the desired yield 607, which can equal the pre-determined portion amount.

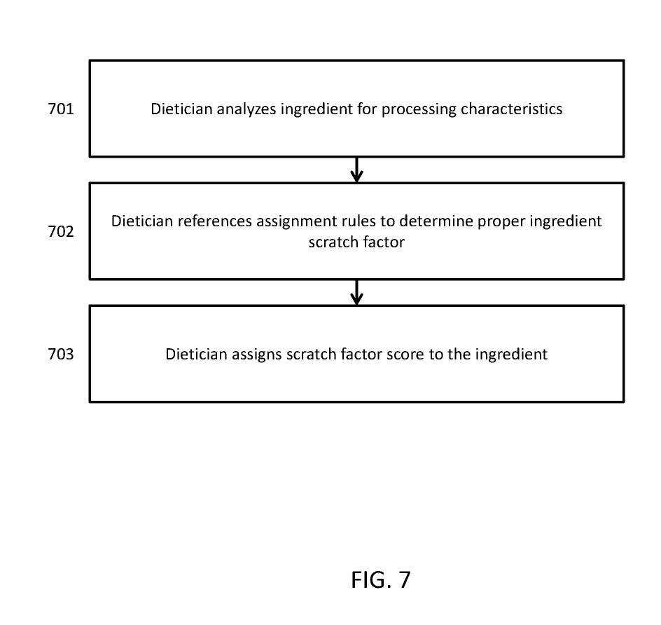

FIG. 7 illustrates a block diagram depicting the process of updating an ingredient scratch factor, in accordance with embodiments described herein. In an embodiment, a dietician can analyze an ingredient for its processing characteristics 701, which can relate to how processed the ingredient is. The dietician can reference one or more scratch factor assignment rules to determine the proper ingredient scratch factor to assign to the ingredient 702. In an embodiment, raw or unprocessed commodities can be assigned the numerical value of one, which can be the highest level of scratch factor. Completely processed foods can be assigned the numerical value of zero. Semi-processed foods can be assigned a value in between one and zero depending on their level of processing. For example, eggs can have a value of one, bread can have a value of zero, and dried pasta, which can require cooking, can receive a value of one half. The dietician can then assign an ingredient scratch factor score to the ingredient 703.

FIG. 8 illustrates an example of a computing environment 800 within which embodiments of the invention may be implemented. Computing environment 800 may be implemented as part of any component described herein. Computing environment 800 may include computer system 810, which is one example of a computing system upon which embodiments of the invention may be implemented. As shown in FIG. 8, the computer system 810 may include a communication mechanism such as a bus 821 or other communication mechanism for communicating information within the computer system 810. The system 810 further includes one or more processors 820 coupled with the bus 821 for processing the information. The processors 820 may include one or more CPUs, GPUs, or any other processor known in the art.

The computer system 810 also includes a system memory 830 coupled to the bus 821 for storing information and instructions to be executed by processors 820. The system memory 830 may include computer readable storage media in the form of volatile and/or nonvolatile memory, such as read only memory (ROM) 831 and/or random access memory (RAM) 832. The system memory RAM 832 may include other dynamic storage device(s) (e.g., dynamic RAM, static RAM, and synchronous DRAM). The system memory ROM 831 may include other static storage device(s) (e.g., programmable ROM, erasable PROM, and electrically erasable PROM). In addition, the system memory 830 may be used for storing temporary variables or other intermediate information during the execution of instructions by the processors 820. A basic input/output system (BIOS) 833 containing the basic routines that help to transfer information between elements within computer system 810, such as during start-up, may be stored in ROM 831. RAM 832 may contain data and/or program modules that are immediately accessible to and/or presently being operated on by the processors 820. System memory 830 may additionally include, for example, operating system 834, application programs 835, other program modules 836 and program data 837.

The computer system 810 also includes a disk controller 840 coupled to the bus 821 to control one or more storage devices for storing information and instructions, such as a magnetic hard disk 841 and a removable media drive 842 (e.g., floppy disk drive, compact disc drive, tape drive, and/or solid state drive). The storage devices may be added to the computer system 810 using an appropriate device interface (e.g., a small computer system interface (SCSI), integrated device electronics (IDE), Universal Serial Bus (USB), or FireWire).

The computer system 810 may also include a display controller 865 coupled to the bus 821 to control a display or monitor 866, such as a cathode ray tube (CRT) or liquid crystal display (LCD), for displaying information to a computer user. The computer system 810 includes a user input interface 860 and one or more input devices, such as a keyboard 862 and a pointing device 861, for interacting with a computer user and providing information to the processor 820. The pointing device 861, for example, may be a mouse, a trackball, or a pointing stick for communicating direction information and command selections to the processor 820 and for controlling cursor movement on the display 866. The display 866 may provide a touch screen interface which allows input to supplement or replace the communication of direction information and command selections by the pointing device 861.

The computer system 810 may perform a portion or all of the processing steps of embodiments of the invention in response to the processors 820 executing one or more sequences of one or more instructions contained in a memory, such as the system memory 830. Such instructions may be read into the system memory 830 from another computer readable medium, such as a hard disk 841 or a removable media drive 842. The hard disk 841 may contain one or more data stores and data files used by embodiments of the present invention. Data store contents and data files may be encrypted to improve security. The processors 820 may also be employed in a multi-processing arrangement to execute the one or more sequences of instructions contained in system memory 830. In alternative embodiments, hard-wired circuitry may be used in place of or in combination with software instructions. Thus, embodiments are not limited to any specific combination of hardware circuitry and software.

As stated above, the computer system 810 may include at least one computer readable medium or memory for holding instructions programmed according to embodiments of the invention and for containing data structures, tables, records, or other data described herein. The term “computer readable medium” as used herein refers to any non-transitory, tangible medium that participates in providing instructions to the processor 820 for execution. A computer readable medium may take many forms including, but not limited to, non-volatile media, volatile media, and transmission media. Non-limiting examples of non-volatile media include optical disks, solid state drives, magnetic disks, and magneto-optical disks, such as hard disk 841 or removable media drive 842. Non-limiting examples of volatile media include dynamic memory, such as system memory 830. Non-limiting examples of transmission media include coaxial cables, copper wire, and fiber optics, including the wires that make up the bus 821. Transmission media may also take the form of acoustic or light waves, such as those generated during radio wave and infrared data communications.

The computing environment 800 may further include the computer system 810 operating in a networked environment using logical connections to one or more remote computers, such as remote computer 880. Remote computer 880 may be a personal computer (laptop or desktop), a mobile device, a server, a router, a network PC, a peer device or other common network node, and typically includes many or all of the elements described above relative to computer 810. When used in a networking environment, computer 810 may include modem 872 for establishing communications over a network 871, such as the Internet. Modem 872 may be connected to system bus 821 via network interface 870, or via another appropriate mechanism.

Network 871 may be any network or system generally known in the art, including the Internet, an intranet, a local area network (LAN), a wide area network (WAN), a metropolitan area network (MAN), a direct connection or series of connections, a cellular telephone network, or any other network or medium capable of facilitating communication between computer system 810 and other computers (e.g., remote computing system 880). The network 871 may be wired, wireless or a combination thereof. Wired connections may be implemented using Ethernet, Universal Serial Bus (USB), RJ-11 or any other wired connection generally known in the art. Wireless connections may be implemented using Wi-Fi, WiMAX, and Bluetooth, infrared, cellular networks, satellite or any other wireless connection methodology generally known in the art. Additionally, several networks may work alone or in communication with each other to facilitate communication in the network 871.

A processor as used herein is a device for executing machine-readable instructions stored on a computer readable medium, for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a computer, controller or microprocessor, for example, and is conditioned using executable instructions to perform special purpose functions not performed by a general purpose computer. A processor may be coupled (electrically and/or as comprising executable components) with any other processor enabling interaction and/or communication there-between. Computer program instructions may be loaded onto a computer, including, without limitation, a general purpose computer or special purpose computer, or other programmable processing apparatus to produce a machine, such that the computer program instructions which execute on the computer or other programmable processing apparatus create means for implementing the functions specified in the block(s) of the flowchart(s). A user interface processor or generator is a known element comprising electronic circuitry or software or a combination of both for generating display elements or portions thereof. A user interface (UI) comprises one or more display elements enabling user interaction with a processor or other device.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters. A graphical user interface (GUI), as used herein, comprises one or more display elements, generated by a display processor and enabling user interaction with a processor or other device and associated data acquisition and processing functions.

The UI also includes an executable procedure or executable application. The executable procedure or executable application conditions the display processor to generate signals representing the UI display images. These signals are supplied to a display device which displays the elements for viewing by the user. The executable procedure or executable application further receives signals from user input devices, such as a keyboard, mouse, light pen, touch screen or any other means allowing a user to provide data to a processor. The processor, under control of an executable procedure or executable application, manipulates the UI display elements in response to signals received from the input devices. In this way, the user interacts with the display elements using the input devices, enabling user interaction with the processor or other device. The functions and process steps herein may be performed automatically or wholly or partially in response to user command. An activity (including a step) performed automatically is performed in response to executable instruction or device operation without user direct initiation of the activity.

A workflow processor, as used herein, processes data to determine tasks to add to, or remove from, a task list or modifies tasks incorporated on, or for incorporation on, a task list, as for example specified in a program(s). A task list is a list of tasks for performance by a worker, user of a device, or device or a combination of both. A workflow processor may or may not employ a workflow engine. A workflow engine, as used herein, is a processor executing in response to predetermined process definitions that implement processes responsive to events and event associated data. The workflow engine implements processes in sequence and/or concurrently, responsive to event associated data to determine tasks for performance by a device and or worker and for updating task lists of a device and a worker to include determined tasks. A process definition is definable by a user and comprises a sequence of process steps including one or more, of start, wait, decision and task allocation steps for performance by a device and or worker, for example. An event is an occurrence affecting operation of a process implemented using a process definition. The workflow engine includes a process definition function that allows users to define a process that is to be followed and may include an Event Monitor.

The system and processes of the figures presented herein are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. Further, the processes and applications may, in alternative embodiments, be located on one or more (e.g., distributed) processing devices on a network linking the units of FIG. 8. Any of the functions and steps provided in the Figures may be implemented in hardware, software or a combination of both. No claim element herein is to be construed under the provisions of 35 U.S.C. 112(f), unless the element is expressly recited using the phrase “means for.”

We claim:

1. A computer implemented method for recipe and menu creation based upon ingredient information, the method comprising:
receiving, from a menu author, one or more menu configuration parameters;
rendering a multi-dimensional grid with cell dimensions corresponding to the one or more menu configuration parameters;
receiving, from the menu author, input for each cell to the multi-dimensional grid specifying one or more recipes, wherein each of the one or more recipes comprise at least one ingredient, each ingredient having an associated ingredient scratch factor corresponding to the ingredient's processing characteristics;
for each of the recipes, calculating a recipe scratch factor based on the associated ingredient scratch factor corresponding to each ingredient in the recipe;
calculating a menu scratch factor by summing the recipe scratch factors;
determining whether the menu meets dietary guidelines; and
if the menu meets the dietary guidelines, displaying the menu to one or more users.

2. The method of claim 1, wherein the multi-dimensional grid is rendered in an HTML user interface.

3. The method of claim 2, wherein a first dimension of the multi-dimensional grid corresponds to meals, a second dimension of the multi-dimensional grid corresponds to days of the week.

4. The method of claim 2, wherein a third dimension of the multi-dimensional grid corresponds to serving stations.

5. The method of claim 1, wherein the recipe scratch factor for each recipe is determined by calculating a weighted average of the associated ingredient scratch factor corresponding to each ingredient in the recipe.

6. The method of claim 1, wherein determining if the menu meets dietary guidelines comprises:
submitting the menu to a dietician for approval; and
receiving feedback from the dietician, wherein the feedback comprises an indication of whether the menu meets dietary guidelines.

7. The method of claim 6, further comprising:
identifying the dietician based on the one or more recipes on the menu.

8. The method of claim 1, wherein determining if the menu meets dietary guidelines comprises:
determining that the menu meets the dietary guidelines if each recipe in the multi-dimensional grid meets or exceeds a corresponding nutritional metric threshold.

9. The method of claim 1, wherein determining if the menu meets dietary guidelines comprises:
determining that the menu meets the dietary guidelines if the recipes in the menu collectively meet or exceed a menu scratch factor minimum value.

10. The method of claim 1, further comprising:
receiving, from the menu author, a request to edit a cell in the multi-dimensional grid;
determining if the recipe in the cell is defined and approved;
receiving, from the menu author, a new recipe to add to the cell; and
in response to receiving the new recipe, recalculating the menu scratch factor.

11. The method of claim 1, wherein the recipe scratch factor is calculated for a recipe using a process comprising:
scaling the ingredients in the recipe to a predetermined portion amount;
calculating the total weight of the recipe;
determining a weight for each ingredient based on the ingredient's percentage of the total weight of the recipe;
weighting the ingredient scratch factor for each ingredient in the recipe based on the weight of the ingredient; and
summing the weighted ingredient scratch factors to produce the recipe scratch factor.

12. The method of claim 11, wherein the process further comprises:
scaling each ingredient's weight to produce a predetermined serving yield.

13. The method of claim 11, wherein the process further comprises:
reducing each ingredient's weight by a preparation loss factor; and
reducing each ingredient's weight by a cooking loss factor.

14. The method of claim 1, further comprising:
receiving, from the menu author, one or more ingredient attributes associated with a new ingredient not used in the one or more recipes in the menu;
receiving a selection, from the menu author, of one or more applicable units of measure for the new ingredient;
determining an ingredient scratch factor for the new ingredient;
storing the new ingredient, the ingredient scratch factor, and an indication that the new ingredient is available for recipe creation.

15. The method of claim 14, wherein ingredient scratch factor for the new ingredient is determined based on input from a dietician.

16. A computer-implemented method for recipe and menu creation based upon ingredient information, the method comprising:
receiving input for each cell to a multi-dimensional grid specifying one or more recipes, wherein each of the one or more recipes comprise at least one ingredient, each ingredient having an associated ingredient scratch factor corresponding to the ingredient's processing characteristics;
for each of the recipes, calculating a recipe scratch factor based on the associated ingredient scratch factor corresponding to each ingredient in the recipe;
calculating a menu scratch factor by summing the recipe scratch factors; and
determining whether the menu meets dietary guidelines; and
if the menu meets the dietary guidelines, saving the menu.

17. A computer-implemented method for recipe and menu creation based upon ingredient information, the method comprising:
creating a menu comprising a plurality of recipes, wherein each recipe comprises at least one ingredient;
for each recipe in the menu, calculating an ingredient scratch factor for each ingredient in the recipe based on the ingredient's processing characteristics;
for each recipe in the menu, calculating a recipe scratch factor based on the ingredient scratch factor corresponding to each ingredient in the recipe;
calculating a menu scratch factor by summing the recipe scratch factors;
determining whether the menu meets dietary guidelines; and
if the menu meets the dietary guidelines, displaying the menu to one or more users.
